(19) United States
(12) Patent Application Publication
(10) Pub. No.: US 2008/0027353 A1
(43) Pub. Date: Jan. 31, 2008

(54) Method of making and using a library of biological information

(76) Inventor: Harvey J. Kliman, Woodbridge, CT (US)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/496,660

(22) Filed: Jul. 31, 2006

(51) Int. Cl. A61B 10/00

(52) U.S. Cl. 600/562

(57) ABSTRACT

Biologic information is obtained concerning a member of a population by obtaining a tissue sample from the member, storing the sample without embedding it in an embedding medium, retrieving from storage the sample associated with the member and thereafter analyzing it for biologic information. The tissue sample may be all or part of the member's placenta. Storage may be in a fixative such as formalin or a formalin substitute. Storage may also be by other means of preserving such as freezing or the like. When a tissue sample from more than one member is collected, a library is created that may be used for a variety of purposes, including reducing the incidence of medical malpractice claims, identification of members such as paternity testing or suspect identification. The library may also be used for pharmaceutical development and epidemiological surveys and research. Each sample may have associated with it certain epidemiologic information such as the donor's identity and medical history, residence, place of employment and the like. If the sample is of placental tissue similar information concerning the member's parents may also be recorded, as well as the hospital where the delivery occurred, the attending physician's name and the like. If and when a sample is retrieved and analyzed for biologic information, that information may also be associated with the sample so that it is available for future researchers.

### CROSS-REFERENCE TO RELATED APPLICATIONS

Not Applicable.

### STATEMENT REGARDING FEDERALLY SPONSORED RESEARCH OR DEVELOPMENT

Not Applicable.

### APPENDIX

Not Applicable

### FIELD OF THE INVENTION

The present invention relates to methods for building and using a library of biological information.

### BACKGROUND OF THE INVENTION

### Abnormal Births and Examination of the Placenta

Each year there are approximately 4 million births in the United States. About twenty percent (˜800,000) of these deliveries have some medical complication (e.g., prematurity, small or large for gestational age, minor congenital anomalies); about 4% (˜160,000) of these children are born with birth defects; about 1-2% (˜40-80,000) have serious complications (e.g., intrauterine fetal demise due to cord accidents or placental abruptions, hypoxic brain damage, severe congenital anomalies, preeclampsia, pregnancy induced hypertension, or bacterial or viral infections). Sadly, about 0.7% (˜28,000) of all infants born in the U.S. die before their first birthday. Since the placenta is part of the fetus, examining it immediately following the delivery of a newborn with medical complications can often reveal the cause of the pregnancy complication or neonatal abnormality. In current medical practice, a pathologist with expertise in anatomic pathology may examine the placenta shortly after the delivery of a newborn with some medical complication and give his findings to the obstetrician, who in turn shares the findings with the parents of the newborn.

Without a placental examination obstetricians often cannot determine the cause of a poor pregnancy outcome, and this leaves families wondering what really happened. Not only is the family left in the dark at the time of the delivery of their affected child, but the physician and family may lose the opportunity to prevent a recurrence of the same complication because they are not informed about what caused the damage in the first place.

This scenario—a poor pregnancy outcome without any explanation of why it occurred—is often fertile ground for litigation against hospitals and medical care givers. The liability costs for such suits are very high. In the case of cerebral palsy, which is only one such example, the costs in the U.S. can easily reach $5 billion/year. This estimate, although large, results from a few simple and reasonable assumptions: an incidence of 3 cases per 1000 deliveries (12,000/year in the U.S.), a litigation rate of 10% of the cases, and an average pay out per case of $4,000,000.

Submitting a placenta for pathologic examination can be the first step in preventing the cycle of accusation and litigation. In cases of poor pregnancy outcome, microscopic examination of the placenta often reveals stresses that may have caused the fetal damage observed in an affected newborn. The major pathologic processes observable in the placenta using current knowledge and techniques that can adversely affect pregnancy outcome include intrauterine bacterial infections, decreased blood flow to the placenta from the mother, and immunologic attack of the placenta by the mother's immune system. Intrauterine infections, most commonly the result of migration of vaginal bacteria through the cervix into the uterine cavity, can lead to severe fetal hypoxia as a result of villus edema (fluid build up within the placenta itself). Both chronic and acute decreases in blood flow to the placenta can cause severe fetal damage and even death. The placenta not only supplies the fetus with nutrition; the placenta is also a barrier between the mother and fetus, protecting the fetus from immune rejection by the mother, a pathologic process that can lead to intrauterine growth retardation or even demise. In addition to these major pathologic categories, many other insults—such as placental separation, cord accidents, trauma, viral and parasitic infections—an adversely impact pregnancy outcome by affecting the function of the placenta.

A trained placental pathologist can examine a placenta and help to explain the causes of a poor pregnancy outcome. A complete placental examination is most useful shortly after the time of delivery when the mother and her affected family are most in need of understanding what happened to their baby. If a full placental examination is not possible at the time of delivery because no placental pathologist is available, then the placenta may be transferred to a center that is prepared to make such an examination. As long as tissue or tissue blocks are saved from the placenta, a microscopic examination of the placenta is always possible at a later time if the need arises.

Today, only a few specialized centers for placental examination exist in the U.S. As the cost of processing and examining placentas decreases, more of the 4 million placentas delivered every year will be able to be examined by appropriately trained physicians. This trend is expected to lead to a better understanding of causes of poor pregnancy outcomes, which in turn is expected to lead to better diagnostic and therapeutic approaches to complicated pregnancies. The ultimate goal of placental examination and research is to insure that babies are healthy.

### Tissue Banks

In 1949 George Hyatt, M.D. established the Navy Tissue Bank, considered to be the first of its kind. Historically, tissue banks stored tissue samples that have been used by the biomedical community for educational and research purposes. More recently, stored tissues have played a major role in the understanding and treatment of diseases such as cancer, HIV/AIDS, and heart disease.

The National Bioethics Advisory Commission (NBAC), established by Executive Order 12975, signed by President Clinton on Oct. 3, 1995, recently reviewed the state of tissue banks. It issued its report “Research Involving Human Biological Materials: Ethical Issues and Policy Guidance (the “NBAC Report”) in January, 2000. Although it was not meant to be a comprehensive inventory, the NBAC Report sought to identify the major sources of stored tissue and to provide information about the policies and procedures utilized by the majority of tissue banks in operation today.

In the NBAC Report, human tissue was defined as including everything from subcellular structures such as DNA, to cells, tissue (bone, muscle, connective tissue, and skin), organs (e.g., liver, bladder, heart, kidney), blood, gametes (sperm and ova), embryos, fetal tissue, and waste (urine, feces, sweat, hair and nail clippings, shed epithelial cells, placentas). The most common source of tissue was noted to be from patients following diagnostic or therapeutic procedures. However, tissue specimens have also been taken during autopsies that are performed to establish cause of death. In addition, tissue has also been available from volunteers who donate blood or other tissue for transplantation or research, organs for transplantation, or their bodies for anatomical studies after death. The authors of the NBAC Report considered placentas to be waste, along with urine and feces.

Based on the Joint Commission on Accreditation of Health Care Organizations (JCAHO), The College of American Pathologist's (CAP) and the Clinical Laboratory Improvement Amendments of 1988 (CLIA-88) standards, as well as common practice, there is no precedent for the long term storage without processing of any tissue, including placentas, that would normally be exempt from pathologic examination. In fact, under current practice these placentas are simply discarded as medical waste (Barry C E. Where do all the placentas go? Can. J. Infect. Control. 1994; 9(1):8-10)—a practice that has raised environmental concerns in some communities (Honolulu Star-Bulletin, Helen Altonn, Jul. 30, 1998).

Examination of the definition of a tissue bank from both government regulatory and scientific view points reveals that placentas have never been considered a component of tissue banks. For example, in the NEW YORK STATE DEPARTMENT OF HEALTH Tissue Resources Program Instructions for Submitting a Request to Licensure As a Human Tissue/Nontransplant Anatomic Bank Pursuant to Subpart 52-2 of 10 NYCRR, New York State defines a tissue bank as follows:
Tissue bank means any person or facility which solicits, retrieves, performs donor selection and/or testing, preserves, transports, allocates, distributes, acquires, processes, stores or arranges for the storage of human tissues for transplantation, transfer, therapy, artificial insemination or implantation, including autogeneic procedures. Tissue banks may be issued a license in the specific category of tissue and type of tissue services and shall be required to comply with the standards applicable to the category or categories of tissue acquired, processed, stored and/or distributed.
Categories of tissue and their definitions are:
Cardiovascular tissue means human heart valves, aorta, great vessels, pericardium, saphenous vein, umbilical vein, or any other cardiovascular tissue for transplantation.
Musculoskeletal tissue means human bone, tendon, ligament, muscle, fascia, cartilage, dura, or any other musculoskeletal tissue for transplantation.
Skin means any human skin tissue for transplantation. Eye means a human cornea or any other ocular tissue for transplantation.
Reproductive tissue means any tissue from the reproductive tract intended for use in artificial insemination or any assisted reproductive procedure. This includes, but is not limited to, semen, oocytes, embryos, spermatozoa, spermatids, ovarian tissue, testicular tissue and epididymal aspirates.
Human milk means human milk for ingestion by a child other than the mother's own.
Hematopoetic progenitor cells means human precursor or progenitor hematopoetic cells derived from bone marrow, peripheral blood or other tissue sources, such as cord blood obtained from the placenta or umbilical cord.

### Although cord blood derived from the placenta or umbilical cord is mentioned, placental tissue not mentioned in these definitions.

Furthermore, the majority of tissue banks that store solid tissues store cancers (see Poster Presentation for ISBER meeting 2005, Seattle Wash. Utilization of Archived Formalin Fixed Paraffin Embedded Tissue for Research. Handorf C R, Kulkarni A L, Pfeffer L M University of Tennessee Health Science Center, Department of Pathology and Laboratory Medicine, Tissue Services Core, Memphis, Tenn., USA), and none of the tissue banks studied in a 2003 Rand Science and Technology survey stored wet tissues (Eiseman E., Brower J., Olmsted S., Clancy N., and Bloom G. (2003). Case Studies of Existing Human Tissue Repositories: “Best Practices” for a Biospecimen Resource for the Genomic and Proteomic Era. RAND Science and Technology). Eiseman et al. found that all the tissue banks examined stored either paraffin-embedded tissue and/or frozen tissue.

There is currently no precedent for saving a sample of a placenta sufficient for subsequent bioanalysis that has not been processed into a block. Currently a placenta is either examined within a few days of delivery, or it is viewed as medical waste and discarded. In the case of the tissue banks cited above, the basis for storage is either future therapeutic use of the tissue (e.g., blood, semen, skin, corneas) or for research purposes (specific cancers, Alzheimer's brains, infected tissues). In both cases the diagnosis is already known. In other words, the tissue is stored with a label as to what it is and why it is being stored. As will become clear in what follows, the present invention deviates from conventional practice by storing tissue samples whether or not they have any specific potential use or pathology.

### The Placenta

The following is a basic explanation of the placenta, its development and common pathologies. In addition, some clinical examples of the benefits of a placental examination are set out as well as a brief explanation of common current practices concerning whether a particular placenta receives a pathologic examination or not.

Definitions and Abbreviations
Amnion the inner layer of the external membranes in direct contact with the amnionic fluid.
Chorion the outer layer of the external membranes composed of trophoblasts and extracellular matrix in direct contact with the uterus.
Chorionic plate the connective tissue that separates the amnionic fluid from the maternal blood on the fetal surface of the placenta.
Chorionic villus the final ramification of the fetal circulation within the placenta.
Cytotrophoblast a mononuclear cell which is the precursor cell of all other trophoblasts.
Decidua the transformed endometrium of pregnancy.
hCG Human chorionic gonadotropin, the main hormone signal of the presence of a pregnancy.
Intervillus space the space between the chorionic villi where the maternal blood circulates within the placenta.
Invasive trophoblast the population of trophoblasts that leave the placenta, infiltrates the endo- and myometrium and penetrates the maternal spiral arteries, transforming them into low capacitance blood channels.
Junctional trophoblast the specialized trophoblasts that keep the placenta and external membranes attached to the uterus.
Spiral arteries the maternal arteries that travel through the myo- and endometrium which deliver blood to the placenta.
Syncytiotrophoblast the multinucleated trophoblast that forms the outer layer of the chorionic villi responsible for nutrient exchange and hormone production.

Formation of the Placenta

A. Early Development

Within a few days of fertilization the embryo develops into a blastocyst, a spherical structure composed on the outside of trophoblasts and on the inside of a group of cells called the inner cell mass. FIG. 1. By 4-5 days after fertilization the embryo (or “blastocyst”) has differentiated into two distinct cell types: an inner cell mass (the lighter cells in FIG. 1)—which will develop into the fetus and eventually become the newborn and trophoblasts (darker cells)—which will develop into the placenta and external membranes. Even by this stage the trophoblasts have begun to make their hallmark hormone: human chorionic gonadotropin (hCG), the hormone that is used as an indicator of a positive pregnancy test. The trophoblasts also mediate the implantation process by attaching to, and eventually invading into the endometrium. FIG. 2.

B. Formation of the Early Placenta

Once firmly attached to the endometrium the developing embryo grows and continues to expand into the endometrium. One of the basic paradigms which is established even within the first week of gestation is that the embryonic/fetal cells are always separated from maternal tissues and blood by a layer of cytotrophoblasts (mononuclear trophoblasts) and syncytiotrophoblasts (multinucleated trophoblasts). FIG. 3.

By nine days the embryo is surrounded by two layers of trophoblasts: the inner mononuclear cytotrophoblasts and the outer multinucleated syncytiotrophoblast layer shown in FIG. 3. This arrangement of embryo, trophoblasts and maternal tissue remains the paradigm throughout gestation. This trophoblast interface not only serves as the means to extract nutrients from the mother, but protects the embryo and fetus from maternal immunologic attack.

At four weeks, the basic structure of the mature placenta has been established: a fetal circulation terminates in capillary loops within chorionic villi which penetrate a maternal blood-filled intervillus space, which, in turn, is supplied by spiral arteries and drained by uterine veins (FIG. 4). The developing chorionic villi remain immersed in a space filled with the nutrient-rich maternal blood. The chorionic villi closest to the maternal blood supply will continue to develop and expand into a mass of chorionic tissue which we identify as the placenta. The chorionic villi farthest away from the maternal blood supply are slowly pushed into the uterine cavity by the expanding amnionic sac which surrounds the embryo. These villi eventually degenerate and form the chorionic layer of the external membranes. At around 20 weeks of gestation the combined amnion-chorion membrane makes contact with the opposite side of the uterus, where it fuses with the decidualized maternal endometrium, forming the complete external membrane consisting of amnion, chorion and decidua layers.

Structure and Function of the Placenta

The placenta is the fetus' extension into the mother, where it functions as the interface between the two. The fetus pumps blood into the placenta via two umbilical arteries that branch over the fetal surface of the placenta. The fetal arteries then dive into the placental mass, continuously branching into units called cotyledons until the blood reaches the capillary loops of the chorionic villi. FIG. 5. New villus branches bud off of the larger villi to increase the mass and exchange surface area of the placenta. The finest branches of the fetal circulation are made up of capillary loops within the chorionic villi. FIG. 6. The fetal circulation branches until it reaches the capillaries of the chorionic villi (Latin for leaf or hair) where exchange of nutrients takes place between the mother and fetus. Once nutrients have been absorbed and waste products released, the fetal blood ultimately collects into the umbilical vein, where it returns to the fetus via the umbilical cord.

Complications of Pregnancy Related to the Placenta

As in any complicated system, problems can arise. Within the scope of this specification it is not possible to discuss all of these pathologic states of the placenta, but the three most important complications of pregnancy related to the placenta are outlined below.

A. Diseases of Trophoblast Invasion: Preeclampsia and Gestational Trophoblastic Neoplasia

Preeclampsia, the clinical state prior to full blown eclampsia (seizures), is one of the ‘toxemias’ of pregnancy. The basic clinical definition of preeclampsia is a “pregnancy-specific condition of increased blood pressure accompanied by proteinuria, edema, or both.” In spite of the simplicity of this description of these clinical signs and symptoms, the etiology of the disease has remained elusive. Many phenomena have been investigated, but the recurring theme appears to be an abnormally low blood flow into the placenta. One of the difficulties has been to distinguish between primary cause and secondary effects. Part of this difficulty may be attributable to the fact that the common end result of low uteroplacental blood flow may be caused by many primary defects. Possibly, therefore, preeclampsia/eclampsia is not a disease, but a syndrome with many causes. Significantly, however, one of the most frequent findings in preeclampsia is decreased or absent trophoblast invasion of the maternal spiral arteries.

Decreased or absent trophoblast invasion may be a consequence of primary defects in the invasive trophoblasts or in the environment that the trophoblasts are attempting to invade. In addition, preeclampsia has been associated with trisomy 13, the chromosome that carries the gene for type IV collagen. Placental bed biopsy in a case of preeclampsia in a multiparous woman carrying a trisomy 13 fetus showed lack of trophoblast invasion of maternal spiral arteries (Feinberg R F, Kliman H J and Cohen A W. (1991) Preeclampsia, Trisomy 13, and the Placental Bed. Obstet Gynecol 78:505-8). These trophoblasts may have had difficulty invading through the maternal extracellular matrix (ECM) because of increased type IV collagen production.

In addition to primary trophoblast defects, many cases of preeclampsia appear to be related to maternal immunologic reaction against the invading trophoblasts. A common clinical finding in these cases is that the invasive trophoblasts have reached the vicinity of the spiral arteries, but have not penetrated them. In addition, the unconverted arteries are often surrounded by lymphocytes, presumably attacking the foreign-appearing invasive trophoblasts. As can be seen from a placental bed biopsy in a typical case of preeclampsia, the invasive trophoblasts have invaded through the endo- and myometrium, but have failed to complete their journey into the spiral arteries (FIG. 7). Failure to convert the maternal spiral arteries into low resistance channels can induce the placenta to secrete vasoactive substances that lead to maternal hypertension. If the maternal blood pressure rises significantly, the spiral arteries can be damaged and may even become occluded, leading to placental infarction.

In contrast to the clinical syndrome of decreased trophoblast invasion, gestational trophoblastic disease (GTD) represents increased and uncontrolled trophoblast invasion. Expanded trophoblast invasion ranges from an exaggerated placental site with increased numbers of benign intermediate trophoblasts (Kurman R J, Main C S, and Chen H C. (1984) Intermediate trophoblast: a distinctive form of trophoblast with specific morphological, biochemical and functional features. Placenta 5:349-69), to placental site trophoblastic tumors, to invasive moles, to frank choriocarcinoma. Morphologic distinction between these forms of trophoblast proliferation can be difficult, but it appears that the normal mechanisms that stop trophoblast invasion are defective in choriocarcinoma cell lines.

B. Infection

More than a third of all preterm births are associated with labor initiated by acute chorioamnionitis (inflammatory infiltrates in the chorionic plate and chorion and amnion layers of the external membranes). Not only does chorioamnionitis have severe consequences for the fetus through the initiation of preterm delivery, but chorioamnionitis increases the risk for cerebral palsy by a factor of at least four.

The Collaborative Perinatal Study (CPS) of the National Institute of Neurological and Communicative Disorders and Stroke followed the course of over 56,000 pregnancies in the United States between 1959 and 1966. In the CPS, more than a third of all preterm births were associated with labor initiated by acute chorioamnionitis. This study also revealed that acute chorioamnionitis was the most frequent cause of stillbirth and neonatal death. Chorioamnionitis not only has severe consequences for the fetus through the initiation of preterm delivery but may—through the initiation of the inflammatory cascade in the placenta and decidua—have direct deleterious effects on the fetus. The CPS showed clearly that acute chorioamnionitis was followed by a 20% greater-than-expected frequency of neurologic abnormalities at 7 years of age.

Infections of the amniotic fluid arise by a variety of routes—including from the abdominal cavity through the fallopian tube, via the maternal blood stream through the placenta, or iatrogenically following amniocentesis or funipuncture—but the most common route is an ascending infection through the cervix. It is not surprising, therefore, that the most common organisms cultured from amnionic fluid are commonly found in the vagina. There are clinical reports, however, of a wide variety of organisms found to cause intrauterine infections, including: Group B streptococci, Listeria monocytogenes, Morganella morganii, Ureaplasma urealyticum, Herpes simplex virus, parvovirus, Chlamydia species, Capnocytophaga, adeno-associated virus, and human immunodeficiency virus. The bacteria that cause intrauterine infections can be found in the amniotic fluid or occasionally within the placental parenchyma itself, while viruses are most often found within the trophoblasts and cells of the villus core.

FIG. 8 shows a cross section of a markedly edematous chorionic villus 16 hours after the initiation of an intrauterine infection. Note the very pale, fluid filled villus core (V). The edema fluid has compressed the fetal vessels (arrows) so severely that only one or two erythrocyte cross sections can be seen in each capillary. The intervillus space is shown at I.

C. Immunologic Rejection

In spite of the fact that the placenta and fetus are ‘foreign’ to the mother, most pregnancies show no evidence of ‘immunologic rejection.’ When immunologic reactions do occur, they can be against any of the components of the gestation (placenta and fetus). These reactions can occur at any stage of pregnancy, and can occur repeatedly, pregnancy after pregnancy.

Although most cases of first trimester pregnancy loss are the result of genetic defects in the fetus and/or placenta, some patients have recurrent pregnancy loss due to repeated maternal immunologic reactions. These reactions can be directed against villus core antigens, against antigens of the syncytiotrophoblast surface—manifested as an intervillositis, or against invasive intermediate trophoblasts. Some practitioners have suggested a variety of therapies for these conditions, including treatment with intravenous immunoglobulins, immunization with paternal or allogenic leukocytes, or exposure to semen through vaginal or rectal suppositories. However, the scientific basis of many of these approaches remains controversial and the efficacy of the therapies proposed has been questioned.

Clinical Examples of Placentas Used in Diagnosis

A placental pathology examination often makes the difference between knowing and not knowing the cause of a poor pregnancy outcome. The following are only a few of the many clinical examples that could be cited to support this contention.

A. Neonatal Death by Disseminated Herpes

An infant was born at 37 weeks of gestation without any apparent problems. However, within a few days he became lethargic. Over the next week he had progressive organ failure and despite aggressive medical intervention he died at 17 days of life. Viral cultures that were collected within a few days of his first becoming sick eventually grew out Herpes simplex virus. Upon learning this the family assumed that the hospital staff had contaminated their child in the newborn nursery, and they sued the hospital and its staff. Fortunately the placenta had been submitted to the hospital's pathology laboratory at the time of the child's birth. During the discovery process the defense counsel solicited the assistance of a placental pathologist to examine the placenta to determine whether the parents' claims had validity. Because the placenta had been submitted to the pathology department at the time of delivery, paraffin blocks were available to make additional slides (called recuts). These recuts were sent to the consulting placental pathologist to be stained by routine methods (hematoxylin and eosin staining) and for immunohistochemistry (IHC) using antibodies against Herpes simplex to determine whether the virus was present in the placental tissues. In this case Herpes simplex was found in the external membranes of the placenta and the umbilical cord. This pattern is diagnostic of an ascending Herpes infection that was initiated while the fetus was still in the uterus. Further questioning of the family revealed that after becoming pregnant the only episode of sexual intercourse occurred two weeks prior to the wife having symptoms of a cold and bloody urine, both classic signs of a primary herpetic infection. The child was born 6 weeks after the episode of intercourse. The case was dropped by the plaintiffs once the results of the placental examination were made available because they confirmed that the infection had in fact occurred prior to the birth and was not caused by the hospital staff. Without the placenta available for examination the true cause and timing of this infection might not have been known.

B. Genetic Basis for Brain Damage

An infant was precipitously born at term. “Precipitous delivery” is a medical term that refers to a rapid delivery without the usual manual assistance of a health care provider. A small subdural hematoma was seen (less than 10 cc of blood under the dura), but otherwise the infant girl appeared to be healthy. However, as the months progressed she failed to meet the standard pediatric milestones, such as sitting up, walking and talking. Eventually the diagnosis of a vegetative brain disorder was made. The only thing that anyone could think of that caused this outcome was the precipitous delivery. Told of the “precipitous delivery” the family concluded this term must imply that the baby girl had flown across the bed at the time of delivery and hit her head on the bed railing with such force that her brain was destroyed. Because the placenta had fortuitously been submitted to the pathology laboratory in this case, the blocks were still available to make recuts for examination. This examination revealed that the placenta had developed in a markedly abnormal fashion, a pattern which indicated that this girl had a genetic disorder that led to her abnormal brain development. The family sued the hospital, but the jury ruled in favor of the defendant because the evidence showed that the placenta demonstrated a cause for the child's brain damage and that she could not have been injured by flying across the bed into the railing at the time of her delivery.

C. False Arrest

The police were called to an apartment because of screaming. Upon entering the apartment they noted drug paraphernalia, several women who appeared to be prostitutes, a man who was possibly a pimp, and a woman in the bathroom with a dead 25 week fetus in the toilet. They arrested the woman with the charge of homicide of a fetus by drugs, presumably cocaine. As she was poor, she languished in the county jail for two years before her case was reviewed by the public defender. Upon reviewing the case the public defender noted that there were no comments about the placenta in the medical examiner's report. She therefore contacted a placental pathologist to determine if the placenta, assuming it was still available, might shed some light on the cause of this 25 week, premature delivery. In fact, microscopic examination of the placenta immediately revealed no evidence of cocaine use, but instead a severe intrauterine infection, the number one cause of preterm labor and delivery in the U.S. Once the public defender shared these findings with the police, the woman was immediately released from jail.

All of these cases have one critical element in common: without a placental examination the true reason for the bad outcome would not have been determined. The fact that the placenta was saved in each of these cases was simply a chance event, because, as was noted above and is discussed more fully below, most placentas are not submitted to pathology, even when there are indications to do so, but especially not in cases when there are no obvious indications. These three cases raise the issue of how long a hospital is obligated to save tissue once it is submitted to pathology. The College of American Pathologists (CAP) guidelines suggest that pathology departments only need to save tissues for a short period of time.

### Standard Practice for Processing and Storing Tissue Samples

The Joint Commission on Accreditation of Healthcare Organizations (JCAHO) standards dictate that hospitals provide for the prompt performance of appropriate examination of all tissue specimens while a patient is under the hospital's care and that this testing be done in either the hospital's laboratories or approved reference laboratories. The JCAHO also requires each hospital to define how such testing will be utilized in each individual's care.

The JCAHO also requires that all specimens, except those identified by the clinical staff as being exempt, be routinely sent to a pathologist for evaluation. The clinical staff, in consultation with a pathologist, decides the exceptions to submitting specimens removed during a surgical procedure to the laboratory. The medical staff and pathologist(s) should approve the tissue exemption list for the institution in writing. Exceptions are made only when the quality of care has not been compromised by the exception, when another suitable means of verifying the surgical removal has been routinely used, and when there is an authenticated operative or other official report that documents the surgical removal.

The CAP Commission On Laboratory Accreditation, Laboratory Accreditation Program Anatomic Pathology Checklist (April 2005), addressed the issue of what tissues are exempt and what tissues must be submitted to the Anatomic Pathology Laboratory. It recommended (see CAP Appendix M) that each institution develop a written policy specifying which specimens need to be submitted to the pathology department and which do not. Furthermore, the Commission stated that this policy should also address which specimens can be submitted, but not examined microscopically.

In routine hospital practice when the clinical findings of a pregnancy or neonatal outcome satisfy the institutionally specified criteria, the placenta from that pregnancy should be sent to the hospital's pathology department for gross and microscopic examination by a trained pathologist. The criteria which might trigger such a placental submission varies from hospital to hospital, but guidelines have been suggested by the College of American Pathologists (CAP). The CAP in 1991 (Althshuler G, Deppisch L M. CAP Conference XIX on the examination of the placenta: Report of the working group on indications for placental examination. Arch Pathol Lab Med. 1991; 115: 701-703) and in 1997 (Langston C, Kaplan C, Macpherson T, et al. Practice guideline for examination of the placenta: developed by the Placental Pathology Practice Guideline Development Task Force of the College of American Pathologists. Archives of Pathology & Laboratory Medicine 1997; 121:449-76) developed a consensus by which placentas should be sent to the hospital pathology laboratory for examination. An example of a compilation of these criteria as currently used at Yale New Haven Hospital follows:
1. All perinatal deaths
2. All stillbirths
3. All therapeutic or spontaneous abortions
4. Mothers with abnormal or high-risk gestations:
   a. Systemic diseases: Diabetes, Lupus, hypertension, ITP, etc.
   b. Genetic diseases
   c. History of intrauterine transfusion or surgery d. History of drug, radiation, toxin or infectious exposure (HIV, CMV, etc.)
5. Abnormal fetus
6. Grossly abnormal placenta or umbilical cord
7. Small or Large for Gestational Age by Obstetrical assessment
8. Preterm birth (<36 weeks) or post-dates (>42 weeks)
9. Birth weight <2,500 or >4,000 grams
10. Abnormal amnionic fluid volume
11. Unexplained or excessive bleeding
12. Thick meconium
13. Multiple births
14. Mothers with recurrent obstetrical complications
15. Clinical chorioamnionitis, suspected neonatal sepsis or ROM >24 hours by obstetrical assessment
16. Patients involved in research protocols or therapeutic trials requiring placental examination.

In spite of this extensive list of inclusion criteria for placental submission, the vast majority of institutions appear to exempt all placentas from pathologic submission. In one study of 413 institutions, Zarbo and Nakhleh (Arch Pathol Lab Med 1999; 123:133-139) found that 66.2% of the institutions exempted placentas from submission and another 13.6% required only a gross examination (total of 79.8% without a microscopic examination). This may explain why in spite of the extensive inclusion criteria listed above, only approximately 10-20% of the potential 4 million placentas delivered each year in the United States are sent to pathology laboratories for examination. In addition to the number of institutions that exempt placentas from submission, it appears that the CAP criteria are not closely followed even when they are policy. Spencer and Khong found in one institution that only one third of the placentas that met the CAP criteria were actually sent to pathology for examination (Spencer M K, Khong T Y. Conformity to guidelines for pathologic examination of the placenta. Archives of Pathology & Laboratory Medicine 2003; 127:205-7).

It is not entirely clear why so few placentas are sent for pathologic examination, but cost may play a role. Currently a placenta submitted to a hospital-based pathology department would be examined grossly (weighed, measured, photographed if unusual, and finally dissected) by either a pathologist or a pathology assistant, and samples taken for histologic processing. Although details of the procedure for examining a placenta may vary from hospital to hospital and pathologist to pathologist, generally the following sequence must be followed. First, a fresh placenta or pieces thereof are fixed in formalin or a formalin substitute. This usually requires at least 12 hours of incubation time. The placenta is visually inspected. It is measured, weighed, and inspected for gross pathology. This gross examination may occur before or after fixing the placenta in formalin.

Next, the placenta is sampled by cutting slices from appropriate places in the placenta, and each slice, about 2 cm.×1 cm.×0.2 cm., is typically put into a plastic cassette. The cassette holds the tissue sample in a small cage, allowing fluids to be circulated around it. Once in the cassette, the tissue sample is subjected to a sequence of chemicals which serve to extract all of the water from the sample and replace the water with paraffin. This is done first by submerging the sample in a mixture of formalin and alcohol. Over time, the mixture of formalin and alcohol in which the cassette is bathed changes until the concentration of alcohol reaches 100%. The bath is then changed to 50% alcohol and 50% xylene, and then through a series of steps until the xylene concentration reaches 100%. The final sequence begins with a bath that is 50% xylene and 50% paraffin. The tissue sample is stepped through a series of baths in which the paraffin concentration is slowly increased to 100% while the xylene concentration is reduced ultimately to zero. The result is a tissue sample in which all the water has been replaced with paraffin. The tissue sample is then placed in a mold with liquid paraffin, which is then allowed to cool. The resulting “block” may be stored, or it may be used to prepare slides for immediate inspection.

To view the tissue sample under a microscope, thin sheets (about 5 micrometers (μm) thick) are cut from the block with a microtome and then mounted on a slide. In some cases, it is necessary to stain the tissue sample in order to reveal details of the cell structures more clearly. To accomplish this, the entire process is reversed until the tissue sample, now mounted on a slide, is again re-hydrated. At that point an appropriate stain is used to highlight certain cell features, as is well known in the art. The entire process for preparing a tissue sample for examination is clearly laborious, expensive and potentially dangerous to the histology workers and to the environment in general, even when using automated equipment to perform the dehydrating and re-hydrating steps.

Because of the issues detailed above, most hospitals have decided that only placentas from cases of obvious pathology are turned over for complete pathologic examination. Otherwise, immediately following the birth, or in some cases after a few days of being held in a refrigerator, the placenta is destroyed. When this happens, if biological information is later needed, there is nothing that can be done. In some hospitals placental tissue samples have been saved as blocks. Generally, this has been done when there is a specific indication such as a child with a birth defect. In one instance a hospital is reported to have saved blocks from all placentas delivered there. (A “block” is a tissue sample that has been processed to replace all the water with paraffin.) However, such practices are rare despite potential advantages. This is apparently because of the high cost to prepare the blocks and because of current government reimbursement policies which allow Medicare coverage only for a pathologic examination that occurs shortly after birth.

This entire process, therefore, is both time-consuming and expensive (˜$500-800). In a capitated environment where insurance only pays a fixed amount for all the healthcare rendered to a pregnant woman, it is not surprising that there is pressure to minimize the number of placental examinations.

In APPENDIX PP entitled “RETENTION OF LABORATORY RECORDS AND MATERIALS” the College of American Pathologists makes the following recommendations for the minimum requirements for the retention of laboratory records and materials. They meet or exceed the regulatory requirements specified in the Clinical Laboratory Improvement Amendments of 1988 (CLIA-88):

| MATERIAL/RECORD | PERIOD OF RETENTION |
|---|---|
| Wet tissue | 2 weeks after final report |
| Paraffin blocks | 10 years |
| Slides | 10 years |
| Reports | 10 years |

### Although some institutions may store their wet tissues for a few weeks more than the recommended 2 week period, it would eventually become prohibitive for hospitals to store placentas for more than a 2-4 week period simply due to space limitations.

In addition to the financial disincentives for placental examination it appears that recent government rulings will lead to even fewer placentas being submitted for pathologic examination. In a Federal case with sentencing on Jun. 10, 2003 in Fort Pierce, Fla., Dr. Leonard Walker, a pathologist, pleaded guilty to health care fraud for performing examinations of placentas that were not medically appropriate. The government accused him of charging the government for examining placentas from normal pregnancies, which the government argued do not need placental examinations, and further that his reports were finalized up to 30 days after the discharge of the infant, at a time, they argued, that the results were no longer clinically relevant to the care of the mother or infant.

This ruling has had a chilling effect on pathology departments around the country. One community hospital in New Haven, Conn., which had previously examined all of the approximately 2,000 placentas delivered each year in its labor and delivery suite, altered its policy so that only placentas that fit very well defined criteria of obvious maternal or neonatal disease could even be submitted for pathologic examination for fear of reprisals that might result from government regulators.

The cumulative effect of all of these forces is that fewer placentas are submitted and examined by pathology departments around the country. This in turn is leading to a loss of critical data that could have helped families understand why their pregnancies had poor or unexpected outcomes. This same trend is also exposing hospitals, physicians and insurance companies to greater liability risk.

### SUMMARY OF DISCLOSURE

Part of what the present invention teaches is preserving in fixative at least a sample of tissue whenever it becomes available and creating a library of two or more unexamined tissue specimens. In the case of placentas, this disclosure teaches preserving placentas from most births and preferably from as many births as possible, and creating a library of unexamined placentas. Thereafter a particular specimen is prepared and examined only when necessary. In following the teachings of this invention, a placenta or other tissue specimen is stored in fixative without any diagnosis, and without any specific anticipated therapeutic need-without a diagnostic label. Part of what the invention teaches is a method of assuring that biological information will be available if and when it is needed. To this end, biologic tissue is sampled, and the sample is prepared and stored in a fixative whenever the tissue becomes available.

Beyond the initial fixation step, the sample is not typically subjected to any of the steps necessary to prepare a slide for examination, and no examination of the tissue sample is performed unless and until it is necessary. The sample is preserved at a very small fraction of the cost of preparing a block or slide, and at an even smaller fraction of the cost of performing a complete pathologic exam. Accordingly, the present invention teaches how to create a library of tissue samples that may be examined individually when a particularized need to do so arises or that may be examined en mass for epidemiologic or other research purposes.

At the present time, cost and clinical justification prevent hospitals from submitting all placentas for pathologic examination. Part of the present invention's teaching is that all (or substantially all) placentas not immediately marked as needing placental examination should be stored intact in a fixative until such time as an examination is necessary. The current placenta paradigm is altered by submitting all or substantially all placentas at a relatively low initial cost without histologic processing or pathologic examination and their associated costs.

Part of the invention's teachings is a series of steps that result in the secure, reliably accessible and confidential storage of placentas in a tissue library. Unlike a tissue bank where the tissue is being saved for potential clinical utilization (such as insemination, transplantation or transfusion) or research purposes (such as analysis of previously diagnosed malignancies, or samples from patients with such diseases as Alzheimer's), this library contains samples that have not been examined and therefore the diagnosis(es) of each sample remains undetermined. The placental tissue library is analogous to a library in which the books have titles and authors (i.e., identifying information) but whose stories are only known when the book is removed, opened and read.

As noted in the Background of the Invention, there are a number of presently appreciated reasons for examining a particular placenta later than in the immediate aftermath of birth. A child may develop a disease that may be treated more effectively after a pathologic examination of the placenta. A placental examination may also assist a family wrestling with determining the cause of a slow developing birth defect or whether to pursue an action alleging medical negligence. Alternatively, a placental examination may be useful to medical personnel faced with defending a lawsuit alleging negligence.

Biologic information may also be useful in other circumstances. For example, DNA testing has been used to identify victims of crime whose bodies are not otherwise identifiable. Because placental tissue includes cells both from the biological mother and from the child, it is possible to deduce those portions of the child's genetic makeup contributed by the father. This information may be helpful in determining paternity as well as, by extension, citizenship (at least where the citizenship of the mother and/or father is known and/or the location of the birth is known). For these reasons, and others, information contained in or derivable from a placenta may prove useful long after birth.

Therefore, the placental library taught by part of this disclosure and the process by which a placenta preserved in fixative would be stored until needed for diagnostic or analytical purposes is novel, unprecedented and non-obvious, especially in light of the fact that current clinical practice is virtually the opposite of this concept. Currently, when a tissue is stored in a tissue bank, there already has been a diagnosis rendered on the tissue itself (which then allows the specimen to be correctly labeled and stored in the tissue bank) or in the absence of such a diagnosis, the patient who is supplying the tissue has a clinical diagnosis which serves to label the specimen as such. Thus, under current practices tissue collections have been screened for common characteristics (such as a particular cancer or a bad pregnancy outcome) and are so grouped.

Accordingly, one of the processes taught herein is a method of obtaining biologic information about a member of a mammalian population comprising plural members. The method comprises steps of:

obtaining a sample of tissue of a member of the population;

holding the sample in storage without embedding it in an embedding medium,

retrieving from storage the sample associated with the selected individual whose biologic information is sought, and thereafter

analyzing the tissue, of the selected individual only when that information is needed.

This disclosure also teaches a method of creating a library of tissue samples of a population comprising the steps of obtaining a sample of tissue from two members of the population and holding the samples for more than ninety-six hours without analyzing them for biologic information.

In addition the disclosure teaches a method of developing a database of genetic information about members of a population comprising the steps of:

obtaining a sample of tissue from two members of the population;

holding the samples without analyzing them for biologic information;

associating with the samples information selected from the group comprising individual information concerning the member who was the source of the sample and population information concerning the member who was the source of the sample;

conducting an analysis of the samples to derive biologic information relating to them; and

associating the information resulting from the biologic analysis with each sample, wherein the step of holding includes a step selected from the group comprising the steps of: (a) storing by placing one of the samples in a container with fixative; (b) storing by placing one of the samples in the freezer; (c) storing by freeze-drying one of the samples; and (d.) storing by placing one of the samples in an inert atmosphere.

For the purposes of this disclosure, “library” refers to a collection of two or more samples. A “sample” is part or all of the available tissue.

### BRIEF DESCRIPTION OF THE DRAWINGS

FIG. 1 shows a blastocyst; the Figure is modified from Sadler T W, Langman's Medical Embryology, 5th edition, Williams & Wilkins, 1985, with permission.

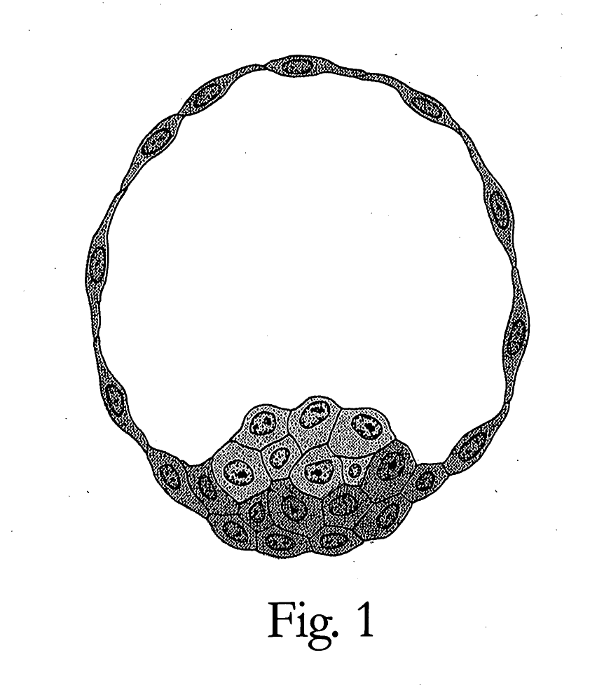

FIG. 2 shows a blastocyst in the process of implanting in the uterine lining, the Figure is modified from Sadler T W, Langman's Medical Embryology, 5th edition, Williams & Wilkins, 1985, with permission.

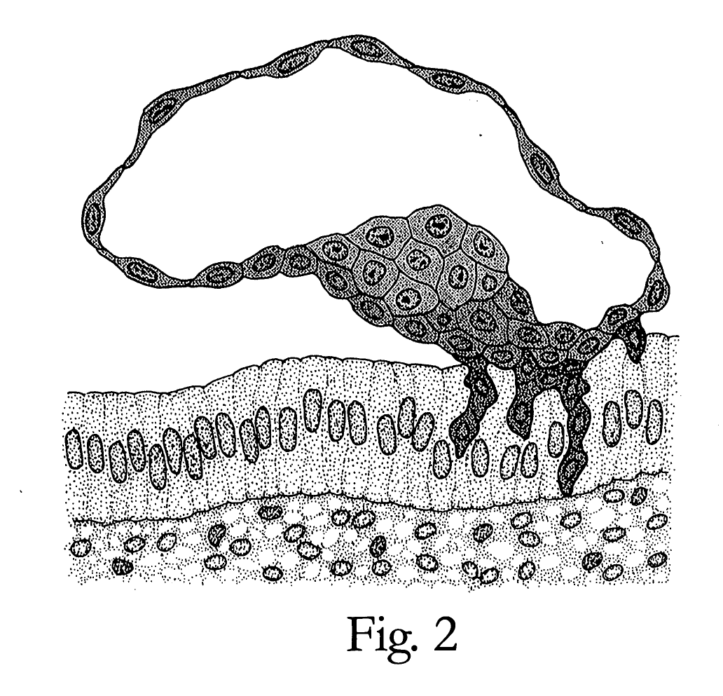

FIG. 3 shows an embryo at about 9 days after conception surrounded by two layers of trophoblasts at the implantation site, the Figure is modified from Sadler T W, Langman's Medical Embryology, 5th edition, Williams & Wilkins, 1985, with permission.

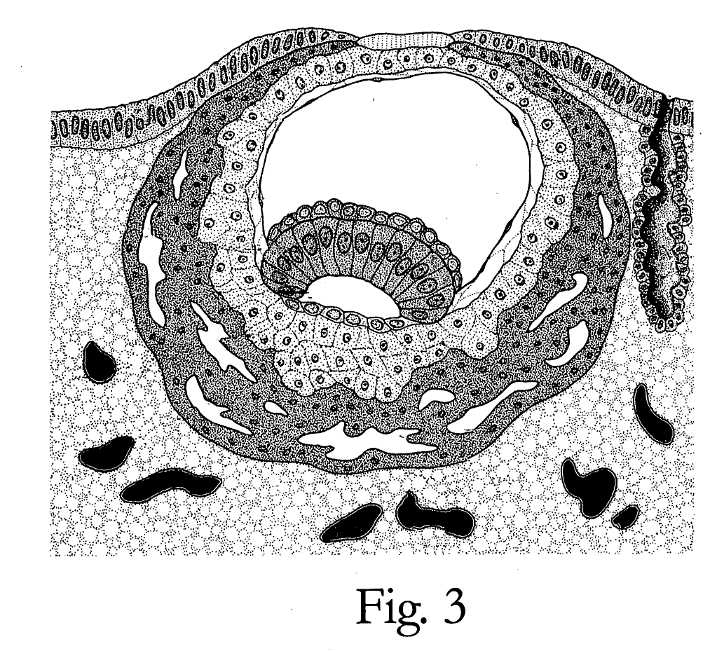

FIG. 4 illustrates an embryo and its implantation site at about four weeks gestation, modified, from Sadler T W, Langman's Medical Embryology, 5th edition, Williams & Wilkins, 1985, with permission.

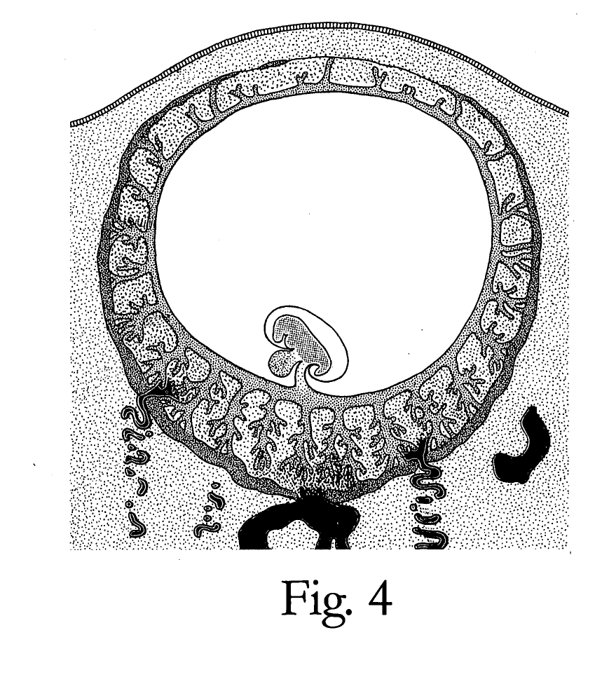

FIG. 5 illustrates maternal and fetal circulations within the placenta, from Moore K L, The developing human, 5th edition, W B Saunders, 1993, with permission.

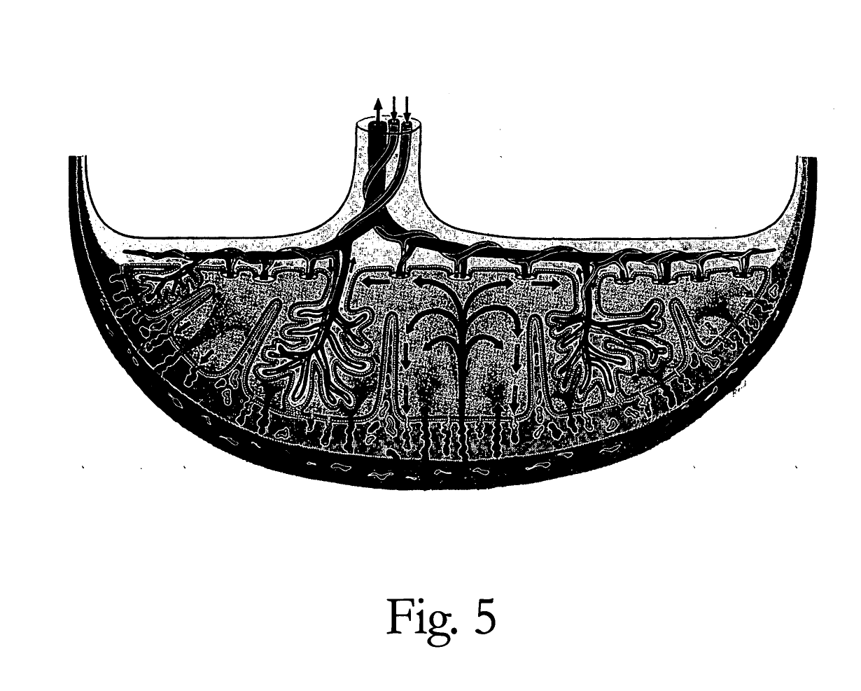

FIG. 6 illustrates terminal chorionic villus, from Sadler T W, Langman's Medical Embryology, 5th edition, Williams & Wilkins, 1985, with permission.

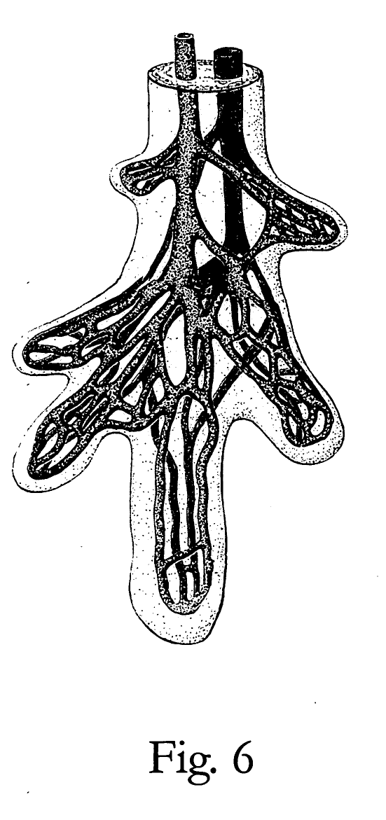

FIG. 7 illustrates a placental bed biopsy of a placental bed from a typical case of preeclampsia showing trophoblasts that have invaded through the endo- and myometrium, but have failed to complete their journey into the spiral arteries.

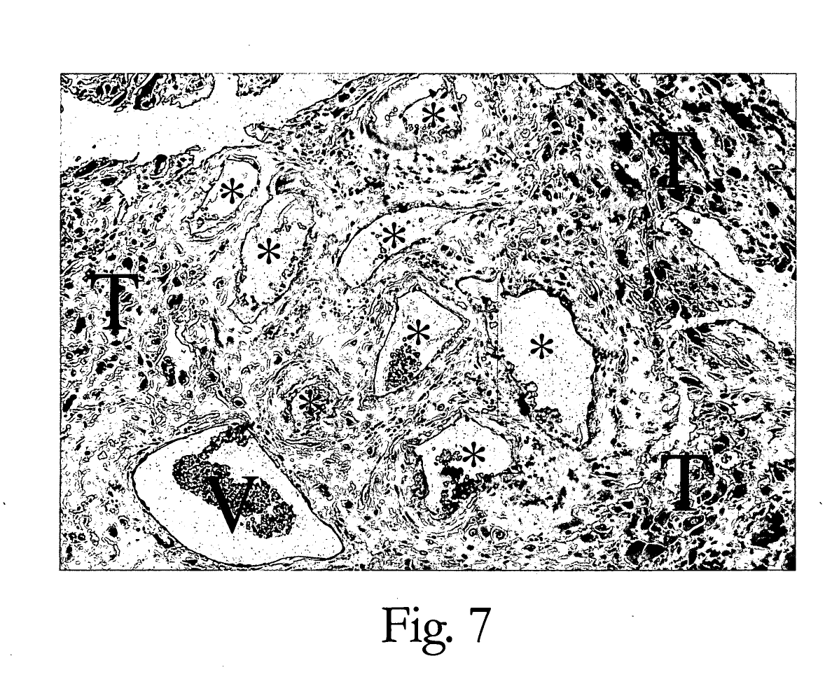

FIG. 8 shows severe villus edema following induction of intrauterine inflammation.

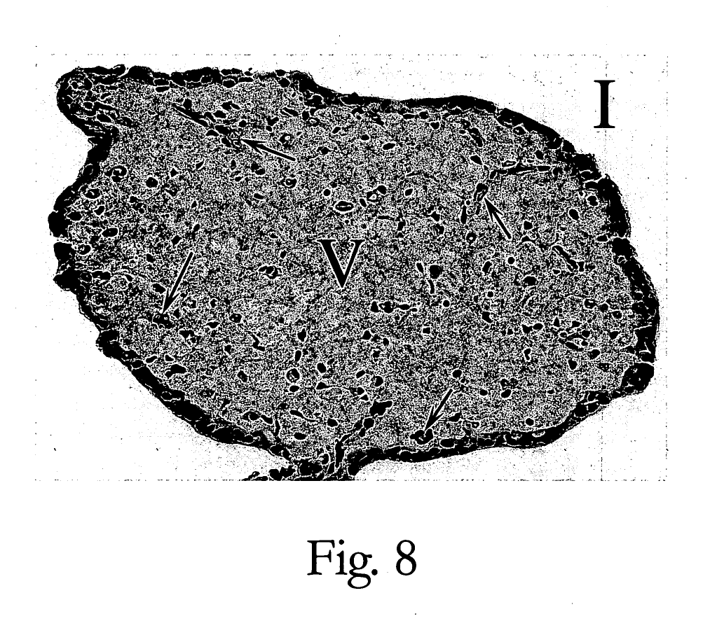

FIG. 9 is a schematic illustration of steps followed in practicing one aspect of the present invention.

### DESCRIPTION OF PREFERRED EMBODIMENTS

Viewing each placenta not as an isolated tissue sample but as a sample from a larger set of samples, a library of placental tissue is created. The library's “collection” grows by saving the placenta or a sample from the placenta of most, if not all, births. The library of placental tissue of a population may then be used for subsequent research and/or diagnosis. In addition to examination focused on learning information about a single individual, the library may also prove useful for epidemiologic or other research purposes. For example, such a library might reveal information concerning so-called “toxic torts,” drug use and effectiveness, and other information derivable from a survey of a large collection of tissue samples. These uses of the library are exemplary; other uses are as unpredictable and numerous as the reasons readers withdraw books from libraries.

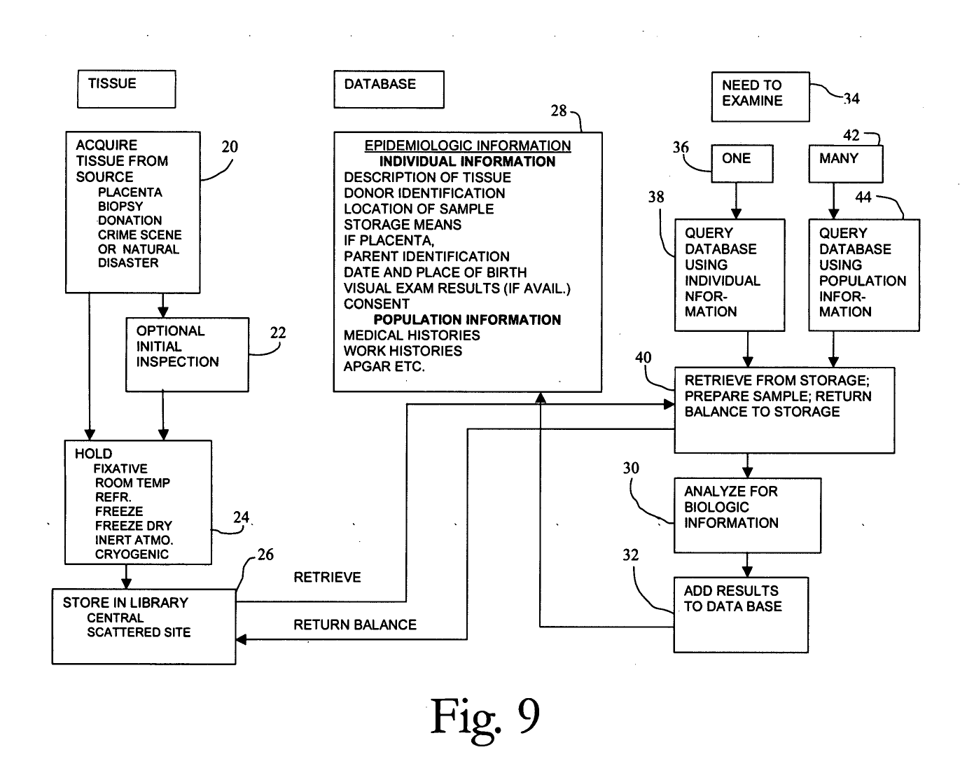

The first step to creating a tissue library is to acquire tissue. This step is illustrated schematically at 20 in FIG. 9. A placenta accompanies each human birth, and these are a source of tissue. A placenta may be weighed, measured and visually inspected at the time of delivery (22 in FIG. 9), although the extent of this analysis may vary, or may even be postponed until the specimen is removed from the library. If the inspection is done at the time of delivery, it is generally done by or under the supervision of the delivering physician or other health professional.

Following the teachings of the present invention after this inspection, if it is performed, the placenta is held for storage as part of the tissue library. Sep 24 in FIG. 9. To do this the placenta can be placed in a container, such as a plastic bucket or zip-loc bag, and kept at room temperature, or preferably refrigerated if it can not be fixed within several hours of the delivery. The placenta can be kept refrigerated for days. At any time during this period from immediately following delivery to several days after delivery, the placenta can be placed in fixative, and this may be done at the hospital where birth takes place, or at a remote processing center. Alternatively, the placenta may be held in a freezer with or without a fixative, it may be freeze-dried, or it may be stored in an inert atmosphere.

If placed in a fixative, one or more among the following may be used: formalin, 10% neutral buffered formalin, formalin substitutes (Glyo-Fixx (Thermo Electron Corp, Pittsburgh, Pa.), STF (Streck Laboratories, Omaha, Nebr.), Omnifix II (FR Chemical Inc, Mount Vernon, N.Y.), Histochoice (Amresco, Solon, Ohio), or Histofix (Trend Scientific, New Brighton, Minn.)), alcohols or as yet to be described or discovered fixatives that allow for both tissue preservation and future analysis. Once placed in fixative the placenta is “fixed” and is stable for as long as there remains liquid fixative around the tissue. For practical purposes such fixed tissue can remain useful and in storage for decades if necessary.

Preparing the tissue samples for storage may be carried out following any one or more of several different strategies. A whole placenta or relatively large pieces of a placenta may be vacuum sealed in a bag with a quantity of formalin or other suitable chemical fixative. Alternatively a smaller piece from each placenta may be stored as a sample, again in a vacuum sealed plastic bag or in a bottle. Other sampling techniques may be used, and other containers may be used. The precise final storage device for each placenta could be one of several options, including plastic containers, plastic bags with a variety of sealing mechanisms, or other containers of shapes and materials that may be optimized for this particular task.

The containers may be stored (step 26 in FIG. 9) at room temperature, at refrigerated temperatures of approximately 10° C., frozen, or cryogenically frozen. In addition, more than one technique for maintaining the library may be used either across the entire library system or even for different samples from the same placenta. Using multiple storage systems may permit additional information to be “read” upon examination of the samples at a future date, and for reasons which may not presently be known. Accordingly, no particular method or methods of storage of the samples are necessary to the practice of this method.

Any of a variety of storage strategies can be employed. These can range from local storage units or facilities at each source (place of deliveries of babies), to centralized facilities that collect specimens from many hospitals or birthing centers. The tissue samples may be stored in a single national warehouse, a series of regional warehouses, or in participating hospitals. A variety of storage facilities may be employed, depending on local conditions, space, transport, economies of scale and population density considerations. The placentas, which are contained in a storage device, are labeled, possibly with a machine readable code or other identifying label, and placed in one of a variety of holding units, for example shelves, boxes, slots, baskets or other optimized solutions for this particular task.

Whenever a tissue sample is collected, information concerning the sample is logged into a database. See step 28 in FIG. 9. Preferably, the database is maintained electronically in a secure, redundant system. However, the data could be maintained on paper, with the records maintained in any convenient manner. Broadly speaking, all the collected data about a particular sample may be regarded as epidemiologic information. The data collected can include several types of information. The initial data collected, termed herein “individual data,” may include a gross description of the tissue, the identity of the donor, the physical location of the tissue, and how it is stored. In the case of a placenta, this information may include the identity of the mother and of her child, the date of birth, the location of the birth, and the results of a visual examination of the tissue if one has been performed, including, for example, its physical dimensions and weight, as well as other readily available information. In addition, consent forms as may be required or appropriate can also be recorded and associated with each sample.

It is expected that additional epidemiologic information will be collected to the extent that it is available. This additional information is termed herein “population data,” and it can include medical histories of the biological mother, of the birth mother if different from the biological mother, and of the father; where they lived and worked both before and during pregnancy, and similar information. Results of any initial examination of the child including APGAR scores may be recorded. The initial information collected (both individual data and population data) does not include biologic information that has been derived from the tissue by pathologic examination because no pathologic examination of the tissue has yet been performed. When and if that information becomes available, it is added to the database to be available to later users of the database. See steps 30 and 32 in FIG. 9. The tissue and the information associated with it thus comprise a database, with some data readily accessible (such as the identifying data and the epidemiologic/demographic data) and some information (such as would be revealed by a pathologic examination of the sample) waiting to be exposed.

When a tissue sample is checked out of the library, only appropriate information is shared. Where the tissue sample is required for an examination related to a particular individual, the full complement of information may be made available. Where a tissue sample is checked out for some other research, certain identifying information may be withheld, consistent with the applicable privacy rights of the mother, father, and child, and/or the needs of the investigators.

At any time, any particular sample will be able to be identified and retrieved if necessary. Any of a variety of data management systems can be employed to accomplish these tasks, such as those that are currently used to log and track express mail packages or to document airline passengers and their luggage as they traverse their respective systems.

There is no minimum period of time for storage of a tissue sample. However, because submission of a placenta for pathologic examination in connection with childbirth ordinarily is initiated while the newborn is still in the hospital, that span provides a useful boundary to distinguish tissue samples stored for archival purposes from those that are merely waiting in a queue for pathologic examination attendant to birth. Thus the present invention contemplates that tissue will be stored for more than three days from delivery (the current norm for a hospital stay incident to a normal, single delivery) and in the case of a longer hospital stay, beyond discharge of the last of the mother and child to leave the hospital, termed herein the “final discharge date.” There are some reports of placental tissue sent for pathologic examination that, in the ordinary course, may not be examined (including report generation) by the pathologist for 72 hours after the final discharge date. It is the intention of this disclosure to distinguish tissue sent for pathologic examination and that is processed routinely from that tissue not previously intended to be examined. Accordingly, this disclosure focuses instead on tissue that under current practice is discarded. The tissue not examined within 96 hours of the final discharge date is outside current routine practices.

While no particular maximum length of time for storing the tissue samples is contemplated, the utility of the library increases as samples are kept longer, and it is contemplated that samples will be preserved for several decades at a minimum. Longitudinal studies, for example, need longitude, and that is achieved only by maintaining the samples for a substantial period of time. In fact, because the cost of storage is relatively slight, presently on the order of fifteen dollars ($15) per year per sample, it is economically feasible to consider storing many generations of tissue samples. On the other hand, if the library is created simply as a defensive legal measure, the custodian may conclude that a statute of limitations on medical malpractice determines the time period beyond which a tissue sample need not be maintained. In any event, it is contemplated that not only will substantially more placental tissue samples be preserved than are currently being preserved, but also that they will be preserved for a substantially longer period of time than the 3 days which under current practices a placenta might remain waiting for examination, thereafter to be discarded as medical waste.

As noted the tissue library includes tissue samples that are preserved in a fixative and then stored along with identifying information. The stored samples may also be frozen, freeze dried, or stored in an inert atmosphere. After a time the need may arise to examine a particular tissue sample or group of tissue samples. Step 34 in FIG. 9. In the event that examination of the tissue sample related to a particular birth is required (step 36), the individual's identifying information is searched (step 38) in the database, and the appropriate tissue sample is removed from storage. At that point a portion of the sample may be checked out of the library for pathologic examination, while returning the remaining tissue to the library for potential future use should it become necessary. See step 40 in FIG. 9. To this end the portion of the tissue sample is processed in the conventional manner, resulting in a tissue sample mounted on a slide suitable for pathologic examination. Alternatively, it may be desirable to examine a group of tissue samples sharing common epidemiologic characteristics as shown schematically at steps 42 and 44 in FIG. 9. In that case, no personal identifying information need be used in selecting the tissue sample(s). Instead, slides or other analyzable materials are prepared in the usual way from one or more samples selected according to the investigator's criteria.

How the retrieved samples are prepared depends on the type of examination required by the circumstances. There are various histological tests which are performed after embedding the specimen in a suitable embedding medium. Suitable embedding media include wax such as paraffin, paramat, paraplast, polyester wax, carbowax polyethylene glycol, and Polyfin®, available from Electron Microscopy Sciences, Hatfield, Pa. Embedding media also include resins such as Araldite, DER, EMbed, JB-4™, Lowicryl, Unicryl™ and Vestpal®, all available from Electron Microscopy Sciences. Embedding media also include freezing media such as TFM™, and Tissue-Tek® O.C.T Compound, also available from Electron Microscopy Sciences. Alternatively, the tissue may require a molecular examination such as a DNA or RNA analysis. It may require chemical analysis such as for drugs, toxins, or other pharmaceutical agents. Such non-histological examinations generally do not require embedding. Because the library collects samples that have not been embedded, they are readily subjected to non-histologic or histologic inspection to obtain biologic information as the demands of the particular case or study may require.

Accordingly, the disclosure contemplates that the present practices for examining tissue, for example a placenta, be modified by preserving a sample of as many placentas as practicable with minimal preparation. The samples are prepared and examined for biologic information only when and as necessary. In this patent application, the data derivable from an external visual examination such as may be performed prior to fixing a tissue specimen is not included within the meaning of the phrase “biologic information”; this phrase is reserved for the kind of information that is derived from a pathologic examination. In this context a “pathologic examination” includes unaided examination of the interior of a tissue sample after it is sliced (bread loafed), microscopic examination of tissue, biochemical and/or molecular tests such as DNA analysis. For convenience in this application these steps are collectively referred to as “pathologic examination.”

In addition to assisting physicians, hospitals, families, insurance companies and lawyers in understanding the cause of a poor pregnancy outcome, the placentas stored in this proposed biological library could also be useful for:
1. As yet to be described diagnostic purposes beyond what is currently accepted Standard Operating Procedure;
2. Identification of the mother, father or the person who was attached to the placenta for civil, criminal or national security purposes;
3. Population studies for pharmaceutical development, targeting and efficacy confirmation;
4. Population studies for genomic targeting and efficacy confirmation;
5. Documentation of identity, place of birth, citizenship;
6. Epidemiologic studies of local groups, regional and national populations with or without identifying information.

Following the teachings of this disclosure, insurance costs would be mitigated, both at the initiation of malpractice suits and in their final resolution. If parents are able to understand why their child has suffered from a poor obstetrical outcome, they are less likely in the first place to seek legal recourse. It has been shown in many studies that one of the major driving forces for bringing a pregnancy-related malpractice case is the family's frustration in not obtaining a clear, concise, unbiased explanation of what occurred in the pregnancy. Since only a portion of poor pregnancy outcomes are observable at the time of delivery, there are many families that, for example, learn that their child has a neurologic abnormality many months after the child's birth. In the absence of a placenta to examine, a major source of elucidation is unavailable to the family. In the case of a library-stored placenta, the discovery of an abnormality in a particular child at, say, 6 months of age would trigger examination of that specific placenta at a time when it has become clinically necessary. The insights gained by this placental examination are likely to yield insights into the causation of the injury, thus answering one of the main concerns for the family. This resolution is often enough to satisfy a family, thereby avoiding resort to legal recourse.

On the other hand, if a case were to come to trial, examination of the placenta at that time is often a critical part of the defense's causation defense. Many of these cases would cease to have the factual support necessary to a plaintiff's verdict. This in turn would dissuade some plaintiff's attorneys from pursuing cases where a placenta is available for examination. Collectively, by both decreasing the number of cases brought to suit and by decreasing the likelihood of a plaintiff's verdict, there would be a decrease in the expenditures of the insurers, which would eventually be translated into decreased fees for the insured.

### Short-Term Retrieval

If the placental sample is held by fixing, it can either be left in the fixative solution for years or it can be removed immediately. In the event that there is a need to examine the sample shortly after it has been placed in fixative, it simply is transported to an appropriate location that can process the sample for a pathologic examination. Once brought to such a facility, the sample is removed from its container, placed on an examination surface (usually a plastic cutting board) and grossly examined. If the sample is an entire placenta, gross examination entails the measurement of the placenta (typically diameters in at least two orthogonal directions, thickness and weight). The weight may be taken before or after the external membranes and umbilical cord are removed, but in either case the state of the placenta at the time of weighing is noted.

During the examination of the placenta it is standard practice for the examiner—typically either through dictation into a recording device or by filling out a checklist—to record the key salient gross distinguishing features of the particular placenta. Alternative ways of recording the observations of the gross examination may be employed as technologies improve in this regard. If necessary to record unusual features, photographs (digital, on film, or via technologies not as yet developed) may be taken of the placenta prior to the commencement of any dissection.

Once this external examination is performed, the examiner will usually bread-loaf the placenta to examine its internal structure. Again, dictation and/or documentation on a checklist may accompany this phase of the examination as is clinically warranted. During the cutting and dissection of the placenta, small (approximately 2×1×0.2 cm) pieces of placenta, external membranes and/or umbilical cord are excised from the whole placenta and placed into cassettes, typically plastic, for processing in a histology laboratory (or any facility that is capable of converting fixed tissues into paraffin embedded tissues or “blocks”). Once processed, the tissues are embedded in an embedding medium such as paraffin or some related wax or an as yet to be developed material and formed into blocks which can be sectioned using a microtome to produce thin sections (typically 5 microns) and which can be placed on glass slides for further processing. This processing typically entails deparaffinization and staining with water soluble dies, such as hematoxylin and eosin, followed by coverslipping (to protect the tissue during examination), and microscopic examination. However, unstained sections can also be utilized for more specialized techniques, such as: immunohistochemistry, in situ hybridization, laser capture microdissection (LCM), and a variety of other specialized tissue analysis techniques in common practice today or as yet to be developed.

### Long-Term Retrieval

Once in fixative in a safe container a placental sample can remain stable for many years. If at any time after the delivery of the placenta there is a need to examine the placenta, it can be retrieved and examined as described above for short-term retrieval. The length of time a particular placenta will need to be stored will be determined by consideration of one or more factors. These could include the applicable statutes of limitation, the desires of the family, medical needs of the child, needs of the health care providers of the child, needs of the hospital or facility where the delivery took place, needs of the local health authorities, needs of state and federal agencies, needs of investigative agencies, needs of local, state or federal security agencies, needs of pharmaceutical or other research organizations, and needs of local, state or federal public health and/or epidemiologic agencies.

### Storage of Non-Placental Materials

Although this application focuses primarily on human placentas, the approaches, uses, applications, techniques and procedures described for placentas can be applied to other human tissues that may not need to be examined at the time of removal. In addition, tissue may also be found at crime scenes, including sites related to genocide or mass murder, or at the locus of humanitarian mass disasters resulting from natural causes such as earthquakes, land slides, or tsunamis. Such tissues could be stored and either examined or utilized for a number of purposes long after their removal in ways similar to the applications described for human placentas.

Moreover, the disclosed methods are not limited to human tissue, and it could be applied to populations of other species of interest. In the context of this disclosure the term “population” means a group of two or more individuals (human or otherwise) Except for marsupials and monotremes, all mammal births include a placenta. While tissue samples from feral animals would not normally be readily available, a tissue library comprising animal tissue could prove a useful research tool. For example, a library of primate tissue might be useful in tracing the evolutionary and or epidemiologic history of HIV or the Ebola virus.

Whenever a pathologic examination of a sample has been completed, the results generally will have an immediate use related to the reason for the examination. A number of examples are described below that illustrate a few of the many reasons a specimen or group of specimens might be studied. According to the teachings of the present invention, the results of the examinations, whatever they may be, may be added to the database. Thus over time, the library will consist of not only the tissue samples, but also pathologic test results of some of the tissue samples. The library's resources thus become richer the more the library is used.

### Prophetic Examples

### Use in Medical Malpractice and Impact on Insurance Costs

In medical malpractice cases involving a damaged infant or stillborn fetus, it is often useful to examine the placenta from the pregnancy to determine the most likely cause for the loss or injury. As described in the clinical examples above, when such an examination reveals the cause of the loss or injury, often a compelling argument can be made which will facilitate resolution of the conflict.

Specific diagnoses do not imply either that medical malpractice existed or not in any particular case. In fact, the exact same diagnosis might be the key for a plaintiff's verdict in one case and the key to a defense verdict in another case. For example, imagine two separate cases in which a newborn has been damaged due to a severe fetal-maternal hemorrhage (a condition in which one of the fetal placental vessels ruptures and causes fetal blood to leak into the maternal circulation). Imagine in both cases that microscopic examination of the placenta reveals clear evidence that a fetal vessel ruptured which resulted in a loss of greater than 80% of its entire blood volume. Imagine in both cases it was noted that the fetus was born pale and lethargic.

At this point the two hypothetical cases diverge. In one case the health care providers recognized immediately that the blood loss occurred and they responded with blood transfusions and volume replacement via intravenous fluid treatment. In spite of the rapid diagnosis and aggressive medical intervention this child was brain damaged because the blood loss had occurred prior to delivery and was not observable until after delivery.

In the second case the health care providers did not assess the newborn to see if he/she had lost any blood until 36 hours after delivery. By that time significant additional damage had occurred which could have been prevented if an immediate intervention had begun shortly after birth. In the law suits that follow, the first case could result in a defense verdict because the jury would be in a position to learn that everything that could have been done was done by the healthcare providers and that they could not be expected to know that a fetal vessel had ruptured in the placenta prior to birth. The second case could produce a plaintiff's verdict because the jury would be in a position to know that the healthcare providers did not appropriately recognize the fact that the newborn had lost a significant amount of its blood and therefore also to know that treatment had been inappropriately delayed.

It has become widely recognized by both sides of medical malpractice litigation that having a qualified examination of the placenta is important before the strength of the case can be appropriately assessed. As in any adversarial relationship, both the plaintiffs' and defendants' attorneys seek ways to escalate their armamentarium prior to negotiating a settlement or taking their case to trial. An important weapon in this process is the determination of causation. Since examination of the placenta often reveals the cause of a poor pregnancy outcome, both sides are increasingly seeking the input of a placental pathologist before they invest thousands of dollars into the workup of their cases. For the plaintiffs attorneys, it is in their best interests to know early on that a case has a cogent defense based on insights gained from a placental examination. In many situations, this will be sufficient to induce their clients to drop rather than pursue a case that they are likely to lose ultimately. For the defense attorneys, knowing the cause of the injury may inspire them either to defend the case at trial, or, if the placenta in fact does not support their theory, settle to case rather than lose at the time of trial.

The cumulative effect of the additional information that placental analysis brings to medical malpractice is predictability. Both parties can assess their respective positions in a more timely fashion and can often make better predictions as to the chances of a favorable verdict. This helps to reduce costs by speeding up the legal process. From the point of view of the insurers, they are better able to manage their short and long term costs and in many cases make better decisions about which cases to defend and which to settle. From the point of view of the plaintiffs' attorneys, insight into causation that a placental examination can provide helps them make wiser decisions as to what cases to take and which to decline.

Finally, parents themselves may choose to have the placentas of their children examined in cases of a delayed poor outcome. If the parents' questions and concerns are fully addressed at the time of this placental examination, then they may not even be motivated to pursue legal remedies, which in turn would have a mitigating effect on the costs of medical malpractice to all parties.

### Use for Identification or Security Purposes

The placenta is a unique tissue. It originally derives from the trophoblast layer of the blastocyst, which is a product of the fertilized egg. Therefore, at its beginning, the placenta, like the newborn itself, is a combination of the genes of the biologic mother and father. Since the mother's blood (which is a mixture of both maternal red blood cells, white blood cells and platelets) circulates in the placenta from the first trimester until the moment of birth, a placenta actually is composed of tissue that genetically matches the newborn (the placental chorionic villi) and the mother (the maternal blood that was left in the placenta at the time of delivery). Therefore, if one were to analyze the DNA (the genetic code) contained in a piece of placenta parenchyma (the soft inner tissue that makes up the bulk of the placenta), one would detect DNA from both the mother and child. By comparing the resultant DNA sequences or patterns (by any number of means that are currently well known or may be developed in the future) with corresponding DNA sequences of either the child or the mother one could:
1. Confirm that the child was once connected to the placenta being tested;
2. Confirm that the stated mother is in fact the mother of the child in question, and/or
3. Deduce what DNA was contributed by the biological father.
   And since the child is a mixture of DNA contributed by their biologic mother and father, if one were to compare the biological father's DNA with the placental DNA sequences or patterns (by any number of means that are currently well known or may be developed in the future), one could:
1. Confirm that the stated father is in fact the biologic father of the child in question; and/or
2. Deduce what DNA was contributed by the biological mother.
   These tools could be utilized in a number of circumstances and regardless of the age of the child. For example:
1. Maternity and paternity testing (to confirm or refute that a particular person is in fact the biologic mother or father),
2. Child confirmation (to confirm or refute that a particular person is in fact the biologic child of a particular mother or father), such as in abduction cases or inadvertent mix-ups in a hospital or birthing center at the time of birth,
3. Forensic identification (the placenta could confirm or refute that a particular sample of DNA was from the person who was once attached to the tested placenta),
4. Citizenship confirmation (the placenta could confirm or refute that a particular sample of DNA was from the person who was once attached to the tested placenta whose birthplace would have been recorded at the time of delivery).

These uses could be put into practice as illustrated below.

### Maternity and Paternity Testing

Today it is common practice to collect a sample of blood from an individual and compare the resultant DNA to the DNA of a child in question to determine if the individual is likely to be the parent of the child (or grown individual) or not. It is equally feasible to do such testing using the placenta itself. This may be necessary in cases where the child is no longer alive, unavailable for testing, or whose identity can not be ascertained. Placental DNA sequences or patterns would be compared to the prospective mother and/or father's DNA sequences or patterns (by any number of means that are currently well known or may be developed in the future). Probabilities would then be generated (or exact matches in cases where DNA sequences are compared directly) as to the likelihood of the tested individuals being the true biologic mother and/or father.

### Child Confirmation

There are unfortunately a number of circumstances where confirmation of child's identity is critical. These include in part cases of abduction, natural disasters where the true biological parent(s) are in question, inadvertent or intended switching of infants at the time of delivery, custody battles, or confirmation of remains in cases of murder, accidental deaths, natural disasters, crime scene analysis or war. In each of these cases, DNA sequences or patterns (by any number of means that are currently well known or may be developed in the future) from the child or remains of the child in question can be compared to the DNA sequences or patterns from the placenta associated with the particular child in question. Probabilities would then be generated (or exact matches in cases where DNA sequences are compared directly) as to the likelihood of the tested individual or remains being associated with the stored placenta (which would have associated with it individual information including the full identity of the individual attached to the placenta).

### Suspect Identification

For hundreds of years forensic scientists and law enforcement agencies have utilized stored forms of identification to associate an individual with a crime or crime scene. Fingerprints have been the major tool utilized in these pursuits, but over the last few decades DNA analysis has risen to prominence as method of identification. Such cases are well known and include both identification of criminals and those falsely accused. There are many examples of cases where the DNA from a crime scene sample was eventually matched to a felon who may have already been in jail or at one time or another given a biological sample that was converted to a stored, searchable DNA source. On the opposite side the Innocence Project has successfully demonstrated many times that the DNA from incarcerated individuals can be used to exonerate falsely accused victims of failures in the judicial system.

A limiting factor in all these cases is having a DNA sample to match to the suspect. In the case of a crime scene where biological material is left behind by the criminal, it is still necessary to match that evidence to a known source of DNA. If a suspect is identified, then his or her DNA can be compared to the crime scene samples. If there is a match, then that will become evidence that the prosecution will likely use to indict the suspect. However, if the suspect's DNA does not match the material left at the crime scene, then a search for other suspects may be necessary. In such cases, testing against the DNA extractable from the stored placentas of potential suspects can easily rule in or out these additional individuals.

### Citizenship Confirmation

The security concerns of the United States of America have changed radically since Sep. 11, 2001. Whereas before that time the U.S. had fairly stringent controls over who entered or stayed in our country, there were many loop-holes that allowed individuals either to enter illegally or to stay beyond their allotted time in this country. Since 9/11 we have been working diligently to increase our security and ensure that only individuals who are either U.S. citizens or are in our country with our permission are here. The difficulty is that we can not be completely sure who is a U.S. citizen. A placental library could help to solve this problem.

For example, if part of the process of creating the birth certificate is to store a placental sample in a placental library, then the identification of an individual as a citizen would be made easier for those whose placenta exists in the placental library. As with a birth certificate, the placental record would contain information concerning the place and time of birth, the parents' names, and other key demographic information. If there were ever a question as to a particular person's citizenship, DNA sequences or patterns from the putative citizen would be compared to the stored placental DNA sequences or patterns (by any number of means that are currently well known or may be developed in the future) to ascertain whether there is a match. In the case were there is no match, citizenship under the identity supplied would not be confirmed. The placenta library could be considered the ultimate identification card.

### Pharmaceutical Development

Humans have been using medicines for thousands of years. Even as long as 6,000 years ago there is clear evidence that ancient Iraqis used pollen grains and flowers to treat diseases, among them marshmallow to sooth mucous membranes, grape hyacinth as a diuretic (increases urination to reduce swelling and the effects of congestive heart failure) and ephedra to relieve asthma. Since that time, much effort has been expended to identify, purify and apply these plant-derived medicinal agents in an effort to improve the human condition. These include Chaulmoogra oil (from Hydnocarpus Gaertn.) used to treat leprosy by Emperor Shen Nung of China around 3000 B.C. and opium poppy and castor oil seed by the Egyptians back to 1500 B.C. The process continues today, as exemplified by the discovery of the potent chemotherapeutic agent taxol, which was first found in fir trees.

Although natural product isolation is the basis of much pharmacology, it has been recently supplemented and replaced by the process of medicinal chemistry, a subset of organic synthesis. Here, chemists either replicate directly the natural products that have been found to be medically effective, or they have gone on to create novel derivatives that may be even more potent or efficacious than the original natural product. We are now entering an even more advanced phase of medicinal development: individualized medicinal fabrication.

As we continue to decipher the human genome we are learning about the differences that make us all unique. One aspect of these distinctions is that we each have a unique set of genes that respond to the environment, and to medicines, in individual ways. For example, a very small subset of patients develop a disease known as malignant hyperthermia in response to general anesthesia. This condition is genetic, appears to be dominant (only one parent need have the trait for it to be passed on to their children), and over 40 specific genes have been identified that are associated with this condition. For these people, general anesthesia induces an often fatal form of overheating. In a similar vein, there is a subset of women who have a certain form of one of the inflammatory cytokines, the compounds that respond to potential infections. For these women, when they develop a minor infection during pregnancy, they have an exaggerated response to the pathogen, which in turn can cause significant damage to them and their pregnancies. Another example are those patients with specific susceptibilities to certain cancers. In many of these families, the existence of particular genes has been associated with increased cancer rates. In all of these cases, and many more that have been described, or have yet to be described, knowledge about the differences between the general population and these individuals is critical to develop individual medications and treatments that are optimized for these people. The key to developing such individualized medicines is to have access to large collections of DNA samples from the general population and from patients with these particular characteristics. A placental library would be an ideal way to provide this information.

By having access to anonymous collections of human tissues, pharmaceutical, genomic and molecular biological researchers, laboratories or companies would have the raw material necessary to identify, understand and potentially to treat or cure a large variety of human diseases—the central goal of all healthcare providers for thousands of years.

### Use for Epidemiology and Population Studies

As with pharmacologic and security uses, a large placental library would have a tremendous impact on studies of populations. As part of the accessioning process of each placenta, specific individual information would be recorded with every placenta. These can include, in part: parent's names, their social security numbers and basic demographics; place of birth; basic findings at birth (gestational age, weight, length, and Apgars if recorded). Additional information might be collected, such as religion, ethnicity, spoken language. Further, a medical history of both the mother and father may be collected and associated with the tissue. If the tissue is other than a placenta, comparable information concerning the donor may also be collected.

Academics, government organizations, and population researchers may wish to analyze selected samples from the placental library based on one or many specific criteria. Access to tissue samples could be associated with very limited, or very extensive associated data. For example, researchers may choose to analyze samples from random placentas with no regard to the epidemiologic data collected. In this case the samples would be completely anonymous. At a more specific level, samples may be analyzed that derive from children born in a specific city, at a specific time, to parents of a specific age or ethnic group. Still deeper analyses may necessitate samples with particular associated medical characteristics, such as specific gestational ages or Apgars. Finally, under very specific situations, selected placentas from individuals may be studied; In this last case, it may be useful to study the placentas from individuals with particular diseases or characteristics that only become obvious some time after birth. For example, children or adults who develop particular diseases, who die of specific medical conditions, or who have traits that researchers wish to study.

What is claimed is:

1. A method of obtaining biologic information about a member of a population, the method comprising the steps of:
obtaining a sample of tissue from a member of the population;
holding in storage the sample without embedding it in an embedding medium;
retrieving from storage the sample associated with the member whose biologic information is sought, and thereafter analyzing the sample for biologic information only when that information is needed.

2. The method of claim 1 wherein the step of obtaining a sample includes the step of obtaining a sample of the member's placental tissue at the time of the member's birth.

3. The method of claim 2 wherein the step of holding includes the step of holding the sample beyond the date of final discharge before analyzing it for biologic information.

4. The method of claim 2 wherein the step of holding includes the step of holding the sample for at least seventy-two hours before analyzing it for biologic information.

5. The method of claim 2 wherein the step of holding includes the step of holding the sample for at least ninety-six hours before analyzing it for biologic information.

6. The method of claim 2 wherein the step of holding includes the step of holding the sample for at least ninety-six hours beyond the date of final discharge before analyzing it for biologic information.

7. The method of claim 2 wherein the step of analyzing includes the step of replacing water in the sample with an embedding medium, wherein said step of replacing is performed only after performing the step of retrieving.

8. The method of claim 1 wherein the step of holding includes the step of associating epidemiologic information with the sample.

9. The method of claim 8 wherein the step of associating includes the step of recording available information comprising the date and location of the member's birth, the identity and medical history of the member's birth mother, the identity and medical history of the member's biologic mother, and the identity and medical history of the member's biologic father.

10. The method of claim 1 wherein the step of retrieving includes the step of associating the sample with information selected from the group comprising: (a) information through which the identity of the member can be determined and (b) information through which the identity of the member cannot be determined.

11. The method of claim 1 wherein the step of holding includes the step of fixing the samples with fixative.

12. The method of claim 1 wherein the step of holding includes the step of storing the samples in a freezer.

13. The method of claim 1 wherein the step of holding includes the step of storing the samples in an inert atmosphere.

14. The method of claim 1 wherein the step of holding includes the step of freeze-drying the samples.

15. A method of creating a library of samples of tissue from a population, the population not having been screened for common characteristics, the method comprising the steps of:
obtaining a sample from two members of the population; and
holding the samples for more than ninety-six hours without analyzing them for biologic information.

16. The method of claim 15 wherein the step of holding comprises a step selected from the group comprising the steps of: (a) storing by placing one of the samples in a container with a fixative; (b) storing by placing one of the samples in a freezer; (c) storing by freeze-drying one of the samples; and (d) storing by placing one of the samples in an inert atmosphere.

17. The method of claim 15 wherein the step of holding includes the step of storing the samples for more than ninety-six hours beyond the date of final discharge without analyzing them for biologic information.

18. The method of claim 15 wherein the step of holding includes the step of fixing the samples in a fixative.

19. The method of claim 18 wherein the fixative is selected from the group comprising formalin, 10% neutral buffered formalin, formalin substitutes, and alcohols.

20. The method of claim 15 wherein the step of holding includes the step of storing the samples at room temperature.

21. The method of claim 15 wherein the step of holding includes the step of storing the samples at or below 10° C.

22. The method of claim 15 wherein the step of holding includes the step of storing the samples at or below 0° C.

23. The method of claim 15 wherein the step of holding includes storing the step of storing the samples in a single storage center.

24. The method of claim 15 wherein the step of holding includes the step of storing the samples in a plurality of storage centers.

25. The method of claim 15 wherein the step of holding includes the step of storing the samples in association with information concerning the member who was the source of the sample selected from the group comprising: (a) individual information and (b) population information.

26. A method of gathering biologic information about members of a population, the method comprising the steps of creating a library according to the method of claim 15,
identifying the members about whom biologic information is sought,
retrieving the sample from the library that is associated with the identified member, and
analyzing the sample to derive biologic information.

27. The method of claim 26 wherein the step of holding includes the step of storing a sample in association with information concerning the identified member selected from the group comprising: (a) individual information and (b) population information, and
the step of retrieving includes the step of retrieving the sample using information associated with the sample selected from the group comprising: (a) individual information and (b) population information.

28. The method of claim 26 wherein the step of retrieving includes the step of retrieving a sample using epidemiologic information that does not include the identity of the member associated with the sample.

29. The method of claim 26 wherein the step of analyzing includes the step of embedding the sample in an embedding medium, wherein the analyzing step follows the step of holding.

30. The method of claim 29 wherein the step of embedding includes the step of embedding the sample in a material selected from the group comprising: tissue freezing medium, wax, and resin.

31. A method of managing liability costs arising from injury to a member of a population comprising the step of creating a library according to the method of claim 25.

32. A library wherein one of the samples is obtained from a library created according to the method of claim 15.

33. A method of determining the cause of a poor pregnancy outcome comprising the steps of:
retrieving a sample from a library created according to the method of claim 15;
thereafter preparing the sample for pathologic analysis; and
conducting a pathologic analysis on the sample.

34. A method of managing medical malpractice liability costs associated with poor pregnancy outcome comprising the steps of:
retrieving samples from a library created according to claim 25, and
determining which poor pregnancy outcomes result from medical malpractice.

35. A method of developing a database of genetic information about members of a population comprising the steps of:
obtaining a sample of tissue from two members of the population;
holding the samples without analyzing them for biologic information;
associating with the samples information selected from the group comprising individual information concerning the member who was the source of the sample and population information concerning the member who was the source of the sample;
conducting an analysis of the samples to derive biologic information relating to them; and
associating the information resulting from the biologic analysis with each sample, wherein the step of holding includes a step selected from the group comprising the steps of: (a) storing by placing one of the samples in a container with a fixative; (b) storing by placing one of the samples in a freezer; (c) storing by freeze-drying one of the samples; and (d) storing by placing one of the samples in an inert atmosphere.

36. A method of creating a library of samples of tissue from a population of a species of mammals, the method comprising:
collecting a sample of the placenta at the births of two members of the population;
collecting epidemiologic data relating to each member whose sample is collected;
associating the epidemiologic data with the sample to which it relates;
holding the epidemiologic data and the sample for four days beyond the final discharge date associated with the birth; and thereafter
analyzing the sample for biologic information,
wherein the step of holding includes a step selected from the group-comprising the steps of: (a) storing by placing the sample in a container with a fixative; (b) storing by placing the sample in a freezer; (c) storing by freeze-drying the sample; and (d) storing by placing the sample in an inert atmosphere

37. The method of claim 36 wherein the step of collecting epidemiologic information includes the step of collecting personal information and the step of collecting population information.

38. The method of claim 36 wherein the step of analyzing includes the step of selecting the sample for analysis using personal information associated with the sample.

39. The method of claim 36 wherein the step of analyzing includes the step of selecting the sample for analysis using population information associated with the sample.

40. A method of managing medical malpractice expenses comprising the step of creating a library according to the method of claim 36, wherein the step of holding includes the step of storing the sample until the expiration of the statute of limitations for malpractice arising from the birth of the member of the population associated with the sample.
